SEQUENCE LISTING (9 sequences)

SEQ ID NO: 1 - DNA, 36 bp, Artificial Sequence
  Description of Artificial Sequence Synthetic primer
actagtcgacatggratggagckggawctttmtctt

SEQ ID NO: 2 - DNA, 36 bp, Artificial Sequence
  Description of Artificial Sequence Synthetic primer
actagtcgacatggawcagacactcctgytatgggt

SEQ ID NO: 3 - DNA, 43 bp, Artificial Sequence
  Description of Artificial Sequence Synthetic primer
actagtcgacatgaggrcccctgctcagwttyttggmwtcttg

SEQ ID NO: 4 - DNA, 29 bp, Artificial Sequence
  Description of Artificial Sequence Synthetic primer
tgcaggacctcaccatgggatggagctgg

SEQ ID NO: 5 - DNA, 31 bp, Artificial Sequence
  Description of Artificial Sequence Synthetic primer
tgactagttgttccttgaccccagtagtcca

SEQ ID NO: 6 - DNA, 29 bp, Artificial Sequence
  Description of Artificial Sequence Synthetic primer
tgcaggacctcaccatgagggcccctgct

SEQ ID NO: 7 - DNA, 20 bp, Artificial Sequence
  Description of Artificial Sequence Synthetic primer
ccttttatttccagcttggt

SEQ ID NO: 8 - DNA, 428 bp, Artificial Sequence
  Description of Artificial Sequence Synthetic polynucleotide
aagcttcaggacctcaccatgggatggagctggatctttctctttctcctgtcaggaactgcaggtgtcctctctgaggttcagctacaacagtctggacctgacctggtgaagcctggggcttcagtgaagatatcctgcaagacttctggatacacattcactgaatacatcatgcactgggtgaagcagagccatggaaagagccttgagtggattggaggtattattcctaacaatggtggtactagctacaaccagaagttcaaggacaaggccacgatgactgtagacaagtcctccagcacaggttacatggaactccgcagcctgacatctgaggattctgcagtctattactgtacaaggcgagaggtgtacgggaggaattactatgctttggactactggggtcaaggaacactagt

SEQ ID NO: 9 - DNA, 406 bp, Artificial Sequence
  Description of Artificial Sequence Synthetic polynucleotide
aagcttcaggacctcaccatgagggcccctgctcagttccttggtctcctgttgctctgttttcaaggtaccagatgtgatatccagatgacacagactacatcctccctgtctgcctctctgggagacagagtcaccatcacttgcagtgcaagtcagggcattaacaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaaccttccgtacacgttcggaggggggaccaagctggaaataaaacgtacgg

(19) United States
(12) Patent Application Publication
(10) Pub. No.: US 2009/0074711 A1
(43) Pub. Date: Mar. 19, 2009

(54) HUMAN THERAPIES USING CHIMERIC AGONISTIC ANTI-HUMAN CD40 ANTIBODY

(75) Inventor: Martin GLENNIE, Southhampton (GB)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: University of Southhampton, Southhampton (GB)

(21) Appl. No.: 11/851,907

(22) Filed: Sep. 7, 2007

Related U.S. Application Data
(60) Provisional application No. 60/842,642, filed on Sep. 7, 2006.

(51) Int. Cl. A61K 38/20; A61K 39/395; A61K 38/21; A61P 35/00

(52) U.S. Cl. 424/85.2; 424/133.1; 424/172.1; 424/85.4; 424/85.7; 424/85.6; 424/178.1

(57) ABSTRACT

Methods of human therapy using a chimeric anti-CD40 antibody, LOB 7/4 or humanized variants thereof, are provided. This CD40 antibody elicits agonistic effects on immunity when used as a monotherapy especially when used in the treatment of human lymphomas and leukemias and other solid tumors In addition, this agonistic CD40 antibody when administered in combination with certain molecules such as TLR agonists or interferons, e.g., alpha and beta interferon, elicits a synergistic effect on immunity.

### FIELD OF THE INVENTION

The invention generally relates to human therapies comprising the administration of a chimeric agonistic anti-human CD40 antibody referred to herein as LOB 7/4 and variants thereof. This chimeric antibody has surprisingly been demonstrated to elicit potent anti-tumor effects on solid CD40 expressing tumors and to potentiate cellular immunity and antitumor effects in humans with advanced cancer. Based on these surprising results the use of this chimeric antibody and variants thereof, e.g., humanized versions thereof as an immune adjuvant or therapeutic for treating various chronic diseases including CD40 expressing cancers, especially solid CD40 expressing tumors as well as its use as an immune adjuvant for treating infectious diseases, autoimmune diseases, allergic and inflammatory diseases is taught. Most preferably, this agonistic anti-human CD40 antibody is used to treat human cancers alone or in combination with other immune potentiators or therapeutic agents, e.g., cytokines, anti-angiogenesis agents, and chemotherapeutics.

### BACKGROUND OF THE INVENTION

It is now widely recognized that the generation of protective immunity depends not only on exposure to antigen, but also the context in which the antigen is encountered. Numerous examples exist in which introduction of a novel antigen into a host in a non-inflammatory context generates immunological tolerance rather than long-term immunity whereas exposure to antigen in the presence of an inflammatory agent (adjuvant) induces immunity. (Mondino et al., Proc. Natl. Acad. Sci., USA 93:2245 (1996); Pulendran et al., J. Exp. Med. 188:2075 (1998); Jenkins et al., Immunity 1:443 (1994); and Kearney et al., Immunity 1:327 (1994)). Since it can mean the difference between tolerance and immunity, much effort has gone into discovering the “adjuvants” present within infectious agents that stimulate the molecular pathways involved in creating the appropriate immunogenic context of antigen presentation.

A molecule known to regulate adaptive immunity is CD40. CD40 is a member of the TNF receptor superfamily and is essential for a spectrum of cell-mediated immune responses and required for the development of T cell dependent humoral immunity (Aruffo et al., Cell 72:291 (1993); Farrington et al., Proc Natl Acad Sci., USA 91:1099 (1994); Renshaw et al., J Exp Med 180:1889 (1994)). In its natural role, CD40-ligand expressed on CD4+ T cells interacts with CD40 expressed on DCs or B cells, promoting increased activation of the APC and, concomitantly, further activation of the T cell (Liu et al Semin Immunol 9:235 (1994); Bishop et al., Cytokine Growth Factor Rev 14:297 (2003)). For DCs, CD40 ligation classically leads to a response similar to stimulation through TLRs such as activation marker upregulation and inflammatory cytokine production (Quezada et al. Annu Rev Immunol 22:307 (2004); O'Sullivan B and Thomas R Crit Rev Immunol 22:83 (2003)) Its importance in CD8 responses was demonstrated by studies showing that stimulation of APCs through CD40 rescued CD4-dependent CD8+ T cell responses in the absence of CD4 cells (Lefrancois et al., J Immunol. 164:725 (2000); Bennett et al., Nature 393:478 (1998); Ridge et al., Nature 393:474 (1998); Schoenberger et al., Nature 393:474 (1998). This finding sparked much speculation that CD40 agonists alone could potentially rescue failing CD8+ T cell responses in some disease settings.

Other studies, however, have demonstrated that CD40 stimulation alone insufficiently promotes long-term immunity. In some model systems, anti-CD40 treatment alone insufficiently promoted long-term immunity. In some model systems, anti-CD40 treatment alone can result in ineffective inflammatory cytokine production. the deletion of antigen-specific T cells (Mauri et al. Nat Med 6:673 (2001); Kedl et al. Proc Natl Acad Sci., USA 98:10811 (2001)) and termination of B cell responses (Erickson et al., J Clin Invest 109:613 (2002)). Also, soluble trimerized CD40 ligand has been used in the clinic as an agonist for the CD40 pathway and what little has been reported is consistent with the conclusion that stimulation of CD40 alone fails to reconstitute all necessary signals for long term CD8+ T cell immunity (Vonderheide et al., J Clin Oncol 19:3280 (2001)).

Both agonistic and antagonistic antibodies specific to CD40 have been suggested to have potential as human therapeutics. Antagonistic anti-CD40 antibodies include those that (1) block CD40/CD40L interaction by at least 90% and have purported antineoplastic properties (Armitage et al., U.S. Pat. No. 5,674,492; Fanslow et al., 1995, Leukocyte Typing V Schlossman et al., eds., 1:555-556); (2) those that antagonize signaling through CD40 (deBoer et al., U.S. Pat. No. 5,677,165) and (3) those that deliver a stimulatory signal through CD40 but do not increase the interaction between CD40 and CD40L, e.g., G28-5, (Ledbetter et al., U.S. Pat. No. 5,182,368; PCT WO 96/18413).

Agonistic anti-CD40 antibodies have been reported by several groups. For example, one mAb, CD40.4 (5C3) (PharMingen, San Diego, Calif.) has been reported to increase the interaction between CD40 and CD40L by approximately 30-40% (Schlossman et al., eds., Leukocyte Typing, 1995, 1:547-556). Additionally, Seattle Genetics in U.S. Pat. No. 6,843,989 allege to provide methods of treating cancer in humans using anti-human CD40 antibodies. These antibodies are alleged to deliver a stimulatory signal, to enhance the interaction between CD40 and CD40L by at least 45% and to enhance CD40L-mediated stimulation and to possess in vivo neoplastic activity. The exemplified antibody disclosed in the Seattle Genetics patent was derived from SC26, an agonistic anti-human CD40 antibody previously shown to deliver strong growth-promoting signals to B lymphocytes (Paulie et al., 1989, J Immunol. 142:590-595).

However, notwithstanding these prior reports, improved methods and human therapies using anti-CD40 antibodies are needed. Particularly, improved methods of treating human cancer and other diseases using anti-human CD40 antibodies which are safe and effective, i.e., which do not elicit undesired side effects but which elicit substantial anti-tumor effects especially on CD40 expressing solid tumors and/or which elicit potent effects on cellular immunity are needed. The present invention satisfies this need and provides other advantages as well.

### SUMMARY OF THE INVENTION

This invention provides novel methods of human treatment using a chimeric anti-human CD40 antibody referred to herein as LOB 7/4 or derivatives thereof, e.g., humanized antibodies or fragments thereof containing the variable heavy and light sequences or CDRs derived from the LOB 7/4 antibody. The present inventors have found that this chimeric antibody possesses advantageous properties when used as a therapeutic, e.g. for treatment of cancer, especially CD40 expressing solid tumors.

It is unexpected that the chimeric antibody used in the present invention would be useful for human therapy. Particularly, it was unpredictable whether such antibody would elicit any adverse side effects in vivo e.g., hepatic toxic effects precluding its usage for human therapy. Indeed, one antibody specific to the ligand for CD40L, humanized 5c8 developed by Biogen (now Biogen IDEC) in collaboration with Columbia University, has been found to cause an adverse incidence of stroke and has been withdrawn from human clinical trials. Also, numerous other antibodies specific to human antigens such as CD4 have been withdrawn from clinical trials because of adverse side effects. Additionally another anti-CD40L antibody was found to elicit hepatic toxicity precluding its use as a therapeutic. (Vonderheide, R. H. et al., J Clin. Oncol. 19(13):3280-7 (2001))

### BRIEF DESCRIPTION OF THE FIGURES

FIG. 1 contains two electrophoretic gels showing the variable light and heavy chains of LOB 7/4.

FIG. 2 contains an electrophoretic gel representing the digestion of the ligated TOPO and LOB 7/4 Vh with the restriction enzymes HIND III/Spel and the ligated TOPO and LOB 7/4 Vk with the restriction enzymes HIND III/BsiWI.

FIG. 3 contains the final consensus sequences of LOB 7/4 Vh and LOB 7/4 Vk.

FIG. 4 shows chimeric LOB 7/4 in pEE6.1, chimeric LOB 7/4 K in pEE12.1 or pEE14.1 all digested with HINDIII and EcoR1 restriction enzymes.

FIG. 5 contains chimeric LOB 7/4H in pEE6.1 and chimeric LOB 7/4k in pEE12.1 or 14.1 all digested with NOT1 and BamH1.

FIG. 6 contains indirect flow cytometric analysis of murine B cells transfected to express human CD40 monoclonal antibody in the supernatant obtained from the transfected CHOK1 cells.

FIG. 7 contains a schematic which represents the method of indirect flow cytometric analysis used to confirm the presence of chimeric anti-CD40 antibody (ch LOB 7/4).

FIG. 8 depicts initial stages in the production of human chimeric anti-CD40 (ch LOB 7/4).

FIG. 9 shows the insertion of chimeric LOB 7/4 heavy and light chains into mammalian expression vectors.

FIG. 10 shows later stages in the development of the chimeric anti-CD40 antibody (ch LOB 7/4)

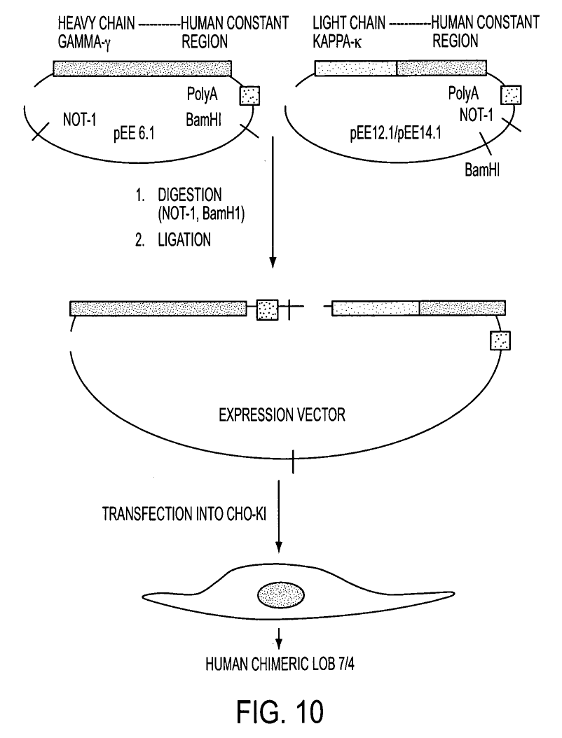

FIG. 11 shows FACS analysis of the early activation marker CD83 on APC subsets (MDC1 myeloid dendritic cells, PDC plasmacytoid dendritic cells and B cells) after whole blood incubation for 4 hours at 37° C./5% CO2, either unstimulated or stimulated with anti-CD40 mAb or CpG oligonucleotide.

FIG. 12. contains an experiment wherein plasma removed from whole blood samples following stimulation with/without anti-CD40 mAb and CpG oligonucleotides was analysed using a multiplex cytokine array panel (Luminex) to determine levels of 10 cytokine/chemokines to assess changes in the plasma cytokine profile.

FIG. 13. contains an experiment wherein Plasma was removed from whole blood samples following stimulation with/without anti-CD40 mAb and CpG oligonucleotides was analysed using a multiplex cytokine array panel (Luminex) to determine levels of 10 cytokine/chemokines to assess changes in the plasma cytokine profile.

### DETAILED DESCRIPTION OF THE INVENTION

The present invention provides novel methods of human therapy by administering an immunologically promoting (adjuvant) or therapeutically effective amount of an anti-human CD40 antibody referred to herein as LOB 7/4, or a variant thereof, or a fragment thereof, especially humanized versions thereof, and/or antibodies or antibody fragments which possess the same epitopic specificity as LOB 7/4 or which competes with LOB 7/4 for binding to human CD40.

More specifically the present invention provides novel methods of treating human cancer, preferably CD40-expressing cancers, and most preferably human CD40 expressing solid tumors by administering a therapeutically effective amount of LOB 7/4 or a fragment, or variant thereof, e.g. a humanized variant. Cancers treatable with the subject CD40 agonistic antibody include by way of example acute leukemia, acute lymphocytic leukemia, acute myelocytic leukemia, myeloblastic promyelocytic myelomonocytic monocytic erythroleukemia, chronic leukemia, chronic myelocytic (granulocytic) leukemia, chronic lymphocytic leukemia, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, and esophageal carcinoma. In preferred embodiments the subject antibody is used to treat CD40 expressing solid tumors such as CD40 expressing melanoma, non-small lung carcinoma, invasive duct breast carcinoma, diffuse large B cell lymphoma, and other solid tumors which express CD40.

Still further the invention provides novel methods of potentiating cellular immunity in a human subject in need of such treatment by administering an amount of LOB 7/4, a variant or fragment thereof, e.g. a humanized version, and/or an anti-human CD40 antibody which competes with and/or binds the same epitope as LOB 7/4 on human CD40 alone or in combination with another active agent such as a cytokine and optionally an antigen. In preferred embodiments this additional agent will comprise a toll like receptor agonist, and will include TLR1, TLR2, TLR3, TLR4, TLR5, TLR6, TLR7, TLR8, TLR9, TLR10 and TLR11 agonists or will comprise a type 1 interferon, particularly alpha or beta interferon. In these preferred embodiments the TLR agonist or cytokine will be administered together or separate from the subject chimeric CD40 agonistic antibody in either order and will be administered in relative amounts that elicit a synergistic effect on immunity, particularly a greater CD8+ T cell cytotoxic response against target cells, e.g., cancer or infected cells than the additive CD8+ T cell cytotoxic response elicited by the antibody and the TLR agonist or cytokine alone. Examples of potential TLR agonists such as CPG oligos, flagellin and synthetic TLR agonists are disclosed in U.S. Ser. No. 10/748,010 incorporated by reference in its entirety herein.

Additionally, the present invention is directed to treating human inflammatory diseases and deficiencies using the subject LOB 7/4 antibody, or fragments, variants thereof and antibodies which bind the same human CD40 epitope or compete with LOB 7/4 for binding to human CD40. These conditions include by way of example systemic lupus erythematosus (SLE), scleroderma (e.g., CRST syndrome), inflammatory myositis, Sjogren's syndrome (SS), mixed connective tissue disease (e.g., MCTD, Sharp's syndrome), rheumatoid arthritis, multiple sclerosis, inflammatory bowel disease (e.g., ulcerative colitis, Crohn's disease) acute respiratory distress syndrome, pulmonary inflammation, osteoporosis, delayed type sensitivity, asthma, primary biliary cirrhosis (PBC), and idiopathic thromboctytopenic purpura (ITP). Again these therapies will include synergistic therapies wherein the subject chimeric antibody is combined with a TLR agonist or cytokine, particularly alpha or beta interferon.

The subject anti-human CD40 antibodies will be administered to a host in need of such treatment in order to elicit an enhanced antitumor or cellular immune response. In preferred embodiments these antibodies will be administered to a subject having or at risk of developing a cancer, an infection, particularly a chronic infectious diseases e.g., involving a virus, bacteria or parasite; or an autoimmune inflammatory or allergic condition. For example the subject antibody can be used to elicit antigen specific cellular immune responses against HIV. HIV is a well recognized example of a disease wherein protective immunity almost certainly will require the generation of potent and long-lived cellular immune responses against the virus.

Thus, this invention provides agonistic antibodies which function as therapeutics or immune adjuvants which can be used in the treatment of chronic infectious diseases involving viruses, bacteria, fungi or parasites as well as proliferative diseases such as cancer, autoimmune diseases, allergic disorders, and inflammatory diseases where effective treatment requires the elicitation of a potent cellular immune response.

As noted, the subject antibodies may be administered in combination with other immune adjuvants such as lymphokines and cytokines and TLR agonists. Examples thereof include interferons such as alpha, beta, and gamma interferon, interleukins such as IL-2, IL-4, IL-6, IL-13 et al., colony stimulating factors, TNFs, and the like.

Additionally, the subject anti-human CD40 antibodies may be administered in combination with other antitumor agents or immune potentiating agents such as chemotherapeutics and cytotoxins commonly used for treating cancer, agents that inhibit angiogenesis, and the like. These additional therapeutic agents may be administered separately or in combination with the subject agonistic anti-CD40 antibody. Also, in some embodiments an effector moiety such as a chemotherapeutic may be directly or indirectly attached to the subject anti-human CD40 antibodies, e.g., by the use of a linker.

Further, in some embodiments the subject anti-human CD40 antibody may be administered in combination with a desired antigen or attached to an antigen which in some instances may act as a targeting moiety.

Exemplary antigens include but are not limited to bacterial, viral, parasitic, allergens, autoantigens and tumor associated antigens. If a DNA based vaccine is used the antigen will be encoded by a sequence the administered DNA construct. Alternatively, if the antigen is administered as a conjugate the antigen will be a protein comprised in the administered conjugate. Still further, the antigen is administered separately from the CD40 antibody and the antigen can take any form. Particularly, the antigen can include protein antigens, peptides, whole inactivated organisms, and the like.

Specific examples of antigens that can be used in the invention include antigens from hepatitis A, B, C or D, influenza virus, Listeria, Clostridium botulinum, tuberculosis, tularemia, Variola major (smallpox), viral hemorrhagic fevers, Yersinia pestis (plague), HIV, herpes, pappilloma virus, and other antigens associated with infectious agents. Other antigens include antigens associated with a tumor cell, antigens associated with autoimmune conditions, allergy and asthma. Administration of such an antigen in conjunction with the subject agonistic anti-CD40 antibody can be used in a therapeutic or prophylactic vaccine for conferring immunity against such disease conditions.

In some embodiments the methods and compositions can be used to treat an individual at risk of having an infection or has an infection by including an antigen from the infectious agent. An infection refers to a disease or condition attributable to the presence in the host of a foreign organism or an agent which reproduce within the host. A subject at risk of having an infection is a subject that is predisposed to develop an infection. Such an individual can include for example a subject with a known or suspected exposure to an infectious organism or agent. A subject at risk of having an infection can also include a subject with a condition associated with impaired ability to mount an immune response to an infectious agent or organism, for example a subject with a congenital or acquired immunodeficiency, an infant, an elderly person, a subject undergoing radiation or chemotherapy, a subject with a burn injury, a subject with a traumatic injury, a subject undergoing surgery, or other invasive medical or dental procedure, or other immunocompromised individual.

Infections which may be treated or prevented using the subject agonistic antibody potentially in combination with other immune potentiators include bacterial, viral, fungal, and parasitic infections. Other less common types of infections also include are rickettsiae, mycoplasms, and agents causing scrapie, bovine spongiform encephalopathy (BSE), and prion diseases (for example kuru and Creutzfeldt-Jacob disease). Examples of bacteria, viruses, fungi, and parasites that infect humans are well know. An infection may be acute, subacute, chronic or latent and it may be localized or systemic. Furthermore, the infection can be predominantly intracellular or extracellular during at least one phase of the infectious organism's agent's life cycle in the host.

Bacterial infections against which the subject antibodies may be used to potentiate a cellular immune response include both Gram negative and Gram positive bacteria. Examples of Gram positive bacteria include but are not limited to Pasteurella species, Staphylococci species, and Streptococci species. Examples of Gram negative bacteria include but are not limited to Escherichia coli, Pseudomonas species, and Salmonella species. Specific examples of infectious bacteria include but are not limited to Heliobacter pyloris, Borrelia burgdorferi, Legionella pneumophilia, Mycobacteria spp. (for example M. tuberculosis, M. avium, M. intracellilare, M. kansaii, M. gordonae), Staphylococcus aureus, Neisseria gonorrhoeae, Neisseria meningitidis, Listeria monocytogeners, Streptococcus pyogenes, (group A Streptococcus), Streptococcus agalactiae(Group B Streptococcus), Streptococcus (viridans group), Streptococcus faecalis, streptococcus bovis, Streptococcus (aenorobic spp.), Streptococcus pneumoniae, pathogenic Campylobacter spp., Enterococcus spp., Haemophilus influenzae, Bacillus anthracis, Corynebacterium diptheriae, Corynebacterium spp., Erysipelothrix rhusiopathie, Clostridium perfringens, Clostridium tetani, Enterobacter aerogenes, Klebsiella pneumoniae, Pasteurella multocida, Bacteroides spp., Fusobacterium nucleatum, Streptobacillus moniliformis, Treponema pallidum, Treponema pertenue, Leptospira, Rickettsia, and Actinomyces israelii.

Examples of viruses that cause infections in humans include but are not limited to Retroviridae (for example human deficiency viruses, such as HIV-1 (also referred to as HTLV-III), HIV-II, LAC or IDLV-III/LAV or HIV-III and other isolates such as HIV-LP, Picornaviridae (for example poliovirus, hepatitis A, enteroviruses, human Coxsackie viruses, rhinoviruses, echoviruses), Calciviridae (for example strains that cause gastroenteritis), Togaviridae (for example equine encephalitis viruses, rubella viruses), Flaviviridae (for example dengue viruses, encephalitis viruses, yellow fever viruses) Coronaviridae (for example coronaviruses), Rhabdoviridae (for example vesicular stomata viruses, rabies viruses), Filoviridae (for example Ebola viruses) Paramyxoviridae (for example parainfluenza viruses, mumps viruses, measles virus, respiratory syncytial virus), Orthomyxoviridae (for example influenza viruses), Bungaviridae (for example Hataan viruses, bunga viruses, phleoboviruses, and Nairo viruses), Arena viridae (hemorrhagic fever viruses), Reoviridae (for example reoviruses, orbiviruses, rotaviruses), Bimaviridae, Hepadnaviridae (hepatitis B virus), Parvoviridae (parvoviruses), Papovaviridae (papilloma viruses, polyoma viruses), Adenoviridae (adenoviruses), Herpeviridae (for example herpes simplex virus (HSV) I and II, varicella zoster virus, pox viruses) and Iridoviridae (for example African swine fever virus) and unclassified viruses (for example the etiologic agents of Spongiform encephalopathies, the agent of delta hepatitis, the agents of non-A, non-B hepatitis (class 1 enterally transmitted; class 2 parenterally transmitted such as Hepatitis C); Norwalk and related viruses and astroviruses).

Examples of fungi include Aspergillus spp., Coccidoides immitis, Cryptococcus neoformans, Candida albicans and other Candida spp., Blastomyces dermatidis, Histoplasma capsulatum, Chlamydia trachomatis, Nocardia spp., and Pneumocytis carinii.

Parasites include but are not limited to blood-borne and/or tissue parasites such as Babesia microti, Babesi divergans, Entomoeba histolytica, Giarda lamblia, Leishmania tropica, Leishmania spp., Leishmania braziliensis, Leishmania donovdni, Plasmodium falciparum, Plasmodium malariae, Plasmodium ovale, Plasmodium vivax, Toxoplasma gondii, Trypanosoma gambiense and Trypanosoma rhodesiense (African sleeping sickness), Trypanosoma cruzi (Chagus' disease) and Toxoplasma gondii, flat worms, and round worms.

As noted this invention preferably is directed to the use of the subject anti-human CD40 antibodies in treating proliferative diseases such as cancers. Cancer is a condition of uncontrolled growth of cells which interferes with the normal functioning of bodily organs and systems. A subject that has a cancer is a subject having objectively measurable cancer cells present in the subjects' body. A subject at risk of developing cancer is a subject predisposed to develop a cancer, for example based on family history, genetic predisposition, subject exposed to radiation or other cancer-causing agent. Cancers which migrate from their original location and seed vital organs can eventually lead to the death of the subject through the functional deterioration of the affected organ. Hematopoietic cancers, such as leukemia, are able to out-compete the normal hematopoietic compartments in a subject thereby leading to hematopoietic failure (in the form of anemia, thrombocytopenia and neutropenia), ultimately causing death.

The antibodies of the invention and synergistic compositions or conjugates containing these antibodies can be used to treat a variety of cancers or subjects at risk of developing cancer, e.g., by the inclusion of a tumor-associated-antigen (TAA). This is an antigen expressed in a tumor cell. Examples of such cancers include breast, prostate, colon, blood cancers such as leukemia, chronic lymphocytic leukemia, and the like. A tumor associated antigen can also be an antigen expressed predominantly by tumor cells but not exclusively.

Additional cancers include those already mentioned as well as basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), lymphoma including Hodgkin's lymphoma and non-Hodgkin's lymphoma; melanoma; neuroblastoma; oral cavity cancer (for example lip, tongue, mouth and pharynx); ovarian cancer; pancreatic cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system; as well as other carcinomas and sarcomas.

The antibodies, compositions containing or conjugates containing the subject antibodies as afore-mentioned can also be used to treat autoimmune diseases such as multiple sclerosis, rheumatoid arthritis, type 1 diabetes, psoriasis or other autoimmune disorders. Other autoimmune disease which potentially may be treated with the vaccines and immune adjuvants of the invention include Crohn's disease and other inflammatory bowel diseases such as ulcerative colitis, systemic lupus eythematosus (SLE), autoimmune encephalomyelitis, myasthenia gravis (MG), Hashimoto's thyroiditis, Goodpasture's syndrome, pemphigus, Graves disease, autoimmune hemolytic anemia, autoimmune thrombocytopenic purpura, scleroderma with anti-collagen antibodies, mixed connective tissue disease, polypyositis, pernicious anemia, idiopathic Addison's disease, autoimmune associated infertility, glomerulonephritis) for example crescentic glomerulonephritis, proliferative glomerulonephritis), bullous pemphigoid, Sjogren's syndrome, psoriatic arthritis, insulin resistance, autoimmune diabetes mellitus (type 1 diabetes mellitus; insulin dependent diabetes mellitus), autoimmune hepatitis, autoimmune hemophilia, autoimmune lymphoproliferative syndrome (ALPS), autoimmune hepatitis, autoimmune hemophilia, autoimmune lymphoproliferative syndrome, autoimmune uveoretinitis, and Guillain-Bare syndrome. Recently, arteriosclerosis and Alzheimer's disease have been recognized as autoimmune diseases. Thus, in this embodiment of the invention the antibody may be administered in combination with a self-antigen against which the host elicits an unwanted immune response that contributes to tissue destruction and the damage of normal tissues.

The antibodies, synergistic combinations thereof, and conjugates containing the subject agonistic anti-CD40 antibodies can also be used to treat asthma and allergic and inflammatory diseases. Asthma is a disorder of the respiratory system characterized by inflammation and narrowing of the airways and increased reactivity of the airways to inhaled agents. Asthma is frequently although not exclusively associated with atopic or allergic symptoms. Allergy is acquired hypersensitivity to a substance (allergen). Allergic conditions include eczema, allergic rhinitis, or coryza, hay fever, bronchial asthma, urticaria, and food allergies and other atopic conditions. An allergen is a substance that can induce an allergic or asthmatic response in a susceptible subject. There are numerous allergens including pollens, insect venoms, animal dander, dust, fungal spores, and drugs.

Examples of natural and plant allergens include proteins specific to the following genera: Canine, Dermatophagoides, Felis, Ambrosia, Lotium, Cryptomeria, Alternaria, Alder, Alinus, Betula, Quercus, Olea, Artemisia, Plantago, Parietaria, Blatella, Apis, Cupressus, Juniperus, Thuya, Chamaecyparis, Periplanet, Agopyron, Secale, Triticum, Dactylis, Festuca, Poa, Avena, Holcus, Anthoxanthum, Arrhenatherum, Agrostis, Phleum, Phalaris, Paspalum, Sorghum, and Bromis.

It is understood that the subject antibodies, antibody containing compositions, and conjugates thereof can be combined with other therapies for treating the specific condition, e.g., infectious disease, cancer or autoimmune condition. For example in the case of cancer the inventive methods may be combined with chemotherapy or radiotherapy.

Methods of making recombinant antibodies according to the invention are well known to those skilled in the art. The examples infra describe production of the subject chimeric anti-human CD40 antibody and provide the complete sequence for the heavy and light chain hereof. The effective amounts of the protein conjugate or DNA can be determined empirically, but can be based on immunologically effective amounts in animal models. Factors to be considered include the antigenicity, the formulation, the route of administration, the number of immunizing doses to be administered, the physical condition, weight, and age of the individual, and the like. Such factors are well known to those skilled in the art and can be determined by those skilled in the art (see for example Paoletti and McInnes, eds., Vaccines, from Concept to Clinic: A Guide to the Development and Clinical Testing of Vaccines for Human Use CRC Press (1999). As disclosed herein it is understood that the subject DNAs or protein conjugates can be administered alone or in conjunction with other adjuvants.

The subject antibodies and antibody conjugates of the invention can be administered locally or systemically by any method known in the art including but not limited to intramuscular, intravenous, intradermal, subcutaneous, intraperitoneal, intranasal, oral or other mucosal routes. Additional routes include intracranial (for example intracisternal, or intraventricular), intraorbital, ophthalmic, intracapsular, intraspinal, and topical administration. The adjuvants and vaccine compositions of the invention can be administered in a suitable, nontoxic pharmaceutical carrier, or can be formulated in microcapsules or a sustained release implant. The immunogenic compositions of the invention can be administered multiple times, if desired, in order to sustain the desired cellular immune response. The appropriate route, formulation, and immunization schedule can be determined by one skilled in the art.

In the methods of the invention, in some instances the antibody or antibody conjugate may be administered in conjunction with one or several antigens or other active agents, e.g., a cytokine or chemotherapeutic. These compositions and active agents containing may be administered separately or in combination in any order that achieve the desired enhancement of cellular immunity. Typically, these compositions are administered within a short time of one another, i.e. within about several hours of one another, more preferably within about a half hour.

In some instances, it may be beneficial to include a moiety on the recombinant antibody which facilitates affinity purification. Such moieties include relatively small molecules that do not interfere with the function of the polypeptides in the conjugate. Alternatively, the tags may be removable by cleavage. Examples of such tags include poly-histidine tags, hemagglutinin tags, maltase binding protein, lectins, glutathione-S transferase, avidin and the like. Other suitable affinity tags include FLAG, green fluorescent protein (GFP), myc, and the like.

The subject antibodies and antibody conjugates containing can be administered with a physiologically acceptable carrier such as physiological saline. The composition may also include another carrier or excipient such as buffers, such as citrate, phosphate, acetate, and bicarbonate, amino acids, urea, alcohols, ascorbic acid, phospholipids, proteins such as serum albumin, ethylenediamine tetraacetic acid, sodium chloride or other salts, liposomes, mannitol, sorbitol, glycerol and the like. The agents of the invention can be formulated in various ways, according to the corresponding route of administration. For example, liquid formulations can be made for ingestion or injection, gels or procedures can be made for ingestion, inhalation, or topical application. Methods for making such formulations are well known and can be found in for example, “Remington's Pharmaceutical Sciences,” 18^{th} Ed., Mack Publishing Company, Easton Pa.

The subject antibodies can be expressed using any vector capable of directing its expression, for example a cell transduced with the vector. Vectors which may be used include by way of example baculovirus, T7 based vectors for use in bacteria, yeast expression vectors, mammalian expression vectors, viral expression vectors, and the like. Viral vectors include retroviral, adenoviral, adeno-associated vectors, herpes virus, simian virus 40, and bovine papilloma virus vectors.

Prokaryotic and eukaryotic cells that can be used to facilitate expression of the subject antibodies include by way of example microbia, plant and animal cells, e.g., prokaryotes such as Escherichia coli, Bacillus subtilis, and the like, insect cells such as Sf21 cells, yeast cells such as Saccharomyces, Candida, Kluyveromyces, Schizzosaccharomyces, and Pichia, and mammalian cells such as COS, HEK293, CHO, BHK, NIH 3T3, HeLa, and the like. One skilled in the art can readily select appropriate components for a particular expression system, including expression vector, promoters, selectable markers, and the like suitable for a desired cell or organism. The selection and use of various expression systems can be found for example in Ausubel et al., “Current Protocols in Molecular Biology, John Wiley and Sons, New York, N.Y. (1993); and Pouwels et al., Cloning Vectors: A Laboratory Manual”: 1985 Suppl. 1987). Also provided are eukaryotic cells that contain and express the subject DNA constructs.

As used herein, the term “antibody” is used in its broadest sense to include polyclonal and monoclonal antibodies, as well as antigen binding fragments thereof. This includes Fab, F(ab′)2, Fd and Fv fragments.

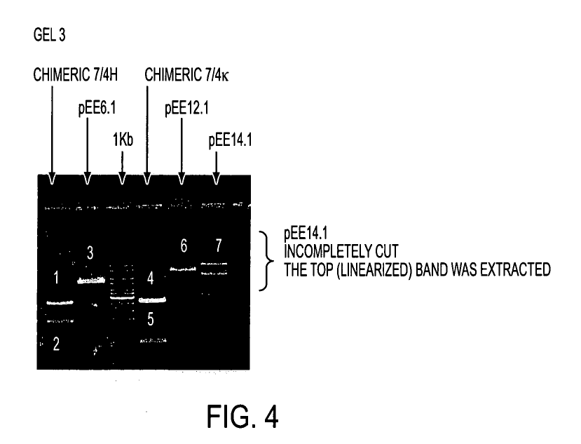

In addition the term “antibody” includes naturally antibodies as well as non-naturally occurring antibodies such as single chain antibodies, chimeric antibodies, bifunctional and humanized antibodies. Preferred for use in the invention are chimeric, humanized and fully human antibodies. Methods for synthesis of chimeric, humanized, CDR-grafted, single chain and bifunctional antibodies are well known to those skilled in the art. In addition, antibodies specific to CD40 are widely known and available and can be made by immunization of a suitable host with a CD40 antigen, preferably human CD40. As noted in the present invention the antibody will comprise chimeric LOB 7/4 having the variable heavy and light chain sequences contained in FIG. 4 the synthesis of which is described in the examples which follow or will comprise a fragment thereof that binds CD40, a variant thereof, e.g., a humanized version, an antibody containing at least the CDRs thereof, or an antibody which competes with and/or binds the same epitope on human CD40 as chimer LOB 7/4 and its murine counterpart (parent LOB 7/4 antibody). While LOB 7/4 had been previously reported in several thesis publications, and in vitro properties thereof including its CDC and ADCC against certain CD40 expressing cells derived from human cancers it was unpredictable whether such antibody would be suitable for use in therapy. Particularly it was uncertain whether this antibody or variants thereof would elicit therapeutic effects in vivo without eliciting adverse side effects precluding its use in human therapy. In particular while the antibody had been tested in a murine model, this model would be inadequate to see if toxicity in humans will result. Also while human xenograft models are helpful as is in vitro data in revealing the cytotoxic properties of an antibody against target cells they are also inadequate to predict whether equivalent results will be observed in humans, e.g., humans who are immunocompromised and/or have advanced cancers and the like wherein CD40 agonistic antibody therapy is intended. Particularly it was unpredictable based on the publicly available information whether the subject chimeric LOB 7/4 antibody when administered at therapeutic dosage amounts, either in a single or multiple dosage regimen would elicit hepatic toxic effects or elicit allergic responses preventing its efficacy. As noted a prior anti-CD40L antibody predicted to be suitable for use in human therapy was found to cause hepatic toxicity in humans. Additionally, it was uncertain whether the subject antibody would elicit adverse effects on kidney function or on the spleen, especially given the animal studies with 3/23 an antibody that binds murine CD40. Still further it was unpredictable whether the antibody will elicit a specific CDC or ADCC response against tumor or other target cells in human patients. Particularly, it was unpredictable whether chimeric LOB 7/4 could be used for treating CD40 expressing cancers in humans.

It is understood that modifications which do not substantially affect the activity of the various embodiments of this invention are also provided within the definition of the invention provided herein. Accordingly, the following examples are intended to illustrate but not limit the present invention.

### EXAMPLES

### Example 1

### Production of a Chimeric Human Monoclonal Antibody 7/4 According to the Invention

Initially a chimeric anti-human antibody was derived from a murine anti-human CD40 antibody referred to as LOB 7/6. After the synthesis thereof, subsequent growth inhibition work using the high grade human B cell line (RL) indicated that another murine anti-human CD40 antibody referred to as LOB 7/4 might be might be more potent in terms of signaling via CD40 than LOB 7/6. The selection and synthesis of this chimeric antibody and some of its in vitro properties is described in detail below.

Antibody Selection

A panel of LOB anti-CD40 antibodies were tested in a system using elutriated monocytes obtained from a normal donor and cultured at a density of 1.5×10⁶/ml in T-165 flasks containing serum free defined media. GMCSF 10 ng/ml and IL4 20 ng/ml were both added to the flasks. Fresh cytokine was subsequently added on days 3 and 6. On day 6 TNFα 50 ng/ml was added to all but one flask (GMCSF/IL4 control flask) and anti-CD40 antibodies were added to all but the control flask at concentrations of 1 and 10 ng/ml. The panel of anti-CD40 antibodies were compared with a known agonistic mouse anti-human CD40 antibody (sc26). After 9 days of culture the dendritic cell phenotype was assessed by flow cytometric analysis.

The results were tabulated and expressed as a percentage of dendritic cells positive for a selected antibody.

Table 1 below shows the dendritic cell phenotype after 9 day culture with a variety of antibodies

| | Flask 1 | Flask 2 | Flask 3 |
|---|---|---|---|
| Staining Ab-FITC | % | % | % |
| MlgG1 | 1.90 | 1.10 | 1.30 |
| Anti-CD14 | 19.3 | 1.10 | 1.50 |
| Anti-CD54 | 98.4 | 99.7 | 99.6 |
| Anti-CD1a | 6.30 | 4.80 | 5.80 |
| Anti-HLA-DR | 84.3 | 98.0 | 98.8 |
| HLA-ABC | 99.9 | 99.8 | 99.6 |
| Anti-CD40 | 98.5 | 95.6 | 74.8 |
| Anti-CD80 | 53.4 | 91.9 | 91.0 |
| Anti-CD86 | 59.7 | 99.1 | 98.4 |
| Anti-CD83 | 8.20 | 87.1 | 89.8 |

Flask 1 - 10 ng/ml GMCSF + 20 ng/ml IL4
Flask 2 - GMCSF/IL4 + day 6 50 ng/ml TNFα and anti-CD40 (s2c6) 1 μg/ml
Flask 3 - GMCSF/IL4 + day 6 50 ng/ml TNFα and anti-CD40 LOB 7/4 1 μg/ml

The majority of dendritic cells (>90%) cultured with LOB 7/4 were up-regulated to express the costimulatory molecules CD80 and CD86. These results were similar to those achieved with the control anti-CD40 antibody sc26. Increasing the concentration of anti-CD40 in the culture system to 10 μg/ml did not significantly influence the results. On the basis of this data LOB 7/4 was chosen for attempted chimerisation. Successful production of this chimeric human anti-CD40 antibody would enable the necessary preclinical in-vitro and toxicology work to commence prior to development of a potential appropriate protocol for a phase I trial of this antibody in the treatment of CD40 expressing human solid tumors.

Production of Chimeric Anti-Human CD40 Monoclonal Antibody According to the Invention

mRNA Preparation

A hybridoma cell colony secreting mouse anti-human CD40 (LOB 7/4) was selected and expanded to obtain 1×10⁷ cells as described in the materials and methods chapter. The mRNA was then purified using the Quickprep micro-mRNA kit (Pharmacia; St Albans, Herts). This system utilizes an oligo(dT) matrix which selectively binds the poly(A) tail of mRNA.

cDNA Preparation

cDNA was prepared from mRNA obtained from the above method, using the First strand cDNA synthesis system (Pharmacia; St Albans, Hefts), under the manufacturers guidelines.

DNA Amplification

It was necessary to first identify the leader and frame-work 4 sequences of both the heavy and light chains of LOB 7/4 so that subsequent cloning could be achieved. The variable regions of both heavy (Vγ) and light chains (Vκ) of the mouse anti-human CD40 (LOB 7/4) were amplified using a family of Vγ and Vκ primers in a polymerase chain reaction (PCR) using Taq polymerase (Promega) and the cDNA was prepared as a template. Twelve heavy and 11 light chain 5′ primers were used all of which included the restriction enzyme site Sal1 (GTCGA) and the initiation codon (ATG). The 5′ primers contained sequences of the whole family of heavy and light chains. Of the 12 heavy chain 5′ primers MHV-7 identified the leader sequence of the heavy chain. The reverse (3′) primer used for the heavy chain was MCγ1, it binds to the hinge region of the heavy chain and enables identification of the frame-work 4 sequence. The leader sequences of the light chains were identified with 2 of the 11 5′ primers MKV-2 and MKV-4. The reverse (3′) primer used for both was MκCR, it binds to the amino acid terminal end of the constant region of the kappa chain.

Preliminary amplification using the above primer pairs yielded amplified PCR bands of 420-450 b.p. when the primer MHV-7 was used for the heavy chain and primers MKV-2 and MKV-4 were used for the light chain.

MHV-7:
5′ ACTAGTCGACATGG (A/G) ATGGAGC (T/G) GGA (A/T)
CTTT (A/C) TCTT 3′
MKV-2:
5′ ACTAGTCGACATGGA (T/A) CAGACACTCCTG (T/C)
TATGGGT 3′
MKV-4:
5′ ACTAGTCGACATGAGG (A/G)CCCCTGCTCAG (A/T) TT
(C/T) TTGG (A/C) (A/T) TCTTG 3′

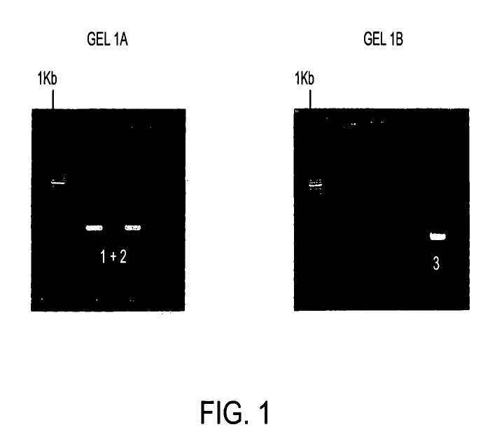

FIG. 1 contains two electrophoretic gels which show the variable light and heavy chain DNA of LOB 7/4.

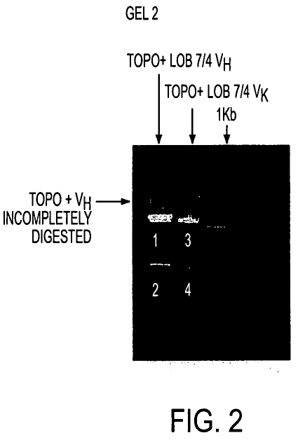

The heavy and light chain DNA was then further amplified using PCR with MHV-7 and MKV-4 primers and Pfu polymerase. MKV-2 was initially utilized but resulted in the detection of an aberrant Vκ chain revealed at the later stage of sequencing. The PCR products were analyzed by agarose gel electrophoresis and visualized under UV light. The bands in the gel representing the PCR fragments of 400 and 420 bp indicated specific amplification of the Vγ and Vκ chains. The bands were extracted from the gel and purified using the QIAEX II agarose gel extraction protocol (Qiagen). The extracted PCR product (100 ng) was ligated with the TOPO blunt II vector (Invitrogen) and transformed into chemically competent Escherichia Coli (E. Coli), TOP-10 cells (Invitrogen) and cultured on plates of agar containing kanamycin. The TOPO-transformants contain a resistance gene to kanamycin, therefore only the appropriately transformed cells grow and establish themselves as a purified colony of the plasmid. The plasmid DNA was then purified from the cultures using QIAprep spin miniprep kit (Qiagen). The purified plasmid DNA was verified by digesting with suitable restriction enzymes (5′ enzyme-Sal I and 3′ enzyme-Xho I) and the presence of the inserted DNA was confirmed using agarose gel electrophoresis. as shown in FIG. 2.

The inserted DNA was then sequenced using T7 and Sp6 primers and the leader sequences aligned so that suitable primers could be designed for subsequent use in the specific amplification of the heavy and light chains of the LOB 7/4. The following primers were then designed. The 5′ primers contained HIND III (AAGCTT) restriction sites, the Kozak sequence (CACCA), and the initiation codon (ATG). The 3′ primers contained restriction enzyme sites Spel (ACTAGT) and BsiWI (CGTACG) for the heavy and light chains respectively. Amplification of the mouse variable regions was then performed using the new primer pairs and cDNA as a template. The DNA obtained was ligated with TOPO blunt II vector as described above and the inserted DNA sequenced.

The designed primers used:

Heavy Chain:

5′: TG CAGGACCTCACCATGGGATGGAGCTGG
3′: TGACTAGTTGTTCCTTGACCCCAGTAGTCCA

Light Chain:

5′: TG CAGGACCTCACCATGAGGGCCCCTGCT
3′: CC TTTTATTTCCAGCTTGGT

The DNA obtained was similarly ligated with TOPO blunt II vector, as described above. Restriction enzyme digests were then performed to confirm the presence of the PCR products within the plasmid (HINDIII, Spel [Vγ], Bsiwl [Vκ]).

FIG. 2 contains an electrophoretic gel representing the digestion of the ligated TOPO and LOB 7/4 V_{H} with the restriction enzymes HIND III/Spel and the ligated TOPO and LOB 7/4 V_{κ}with the restriction enzymes HIND III/BsiWI.

The DNA was then sequenced using T7 and T6 primers as described above.

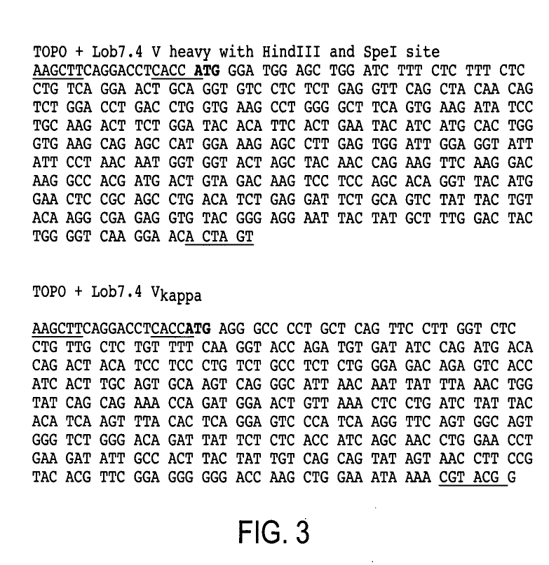

FIG. 3 contains the consensus sequences of LOB 7/4 V_{H} and LOB 7/4 V_{K}.

Chimerisation

The confirmed V_{H} and V_{κ} chains in TOPO blunt II vector were digested with HINDIII/Spel and HINDIII/BsiWI restriction enzymes respectively. The digested variable regions were then ligated with pre-digested pUC plasmids containing either the human heavy chain constant region (pUCγ) or the human kappa chain constant region (pUCκ) to form the chimeric heavy and light chain products. The ligation mixture was then used to transform competent E. Coli JM109 cells. The transformants contain the ampicillin resistance gene and were, therefore, selected to grow when cultured on ampicillin containing agar plates. The presence of the chimeric DNA construct was confirmed by performing a restriction enzyme digest and gel analysis (HINDIII and EcoRI) as shown in FIG. 4.

For stable expression of the chimeric antibody the chimerised heavy and light chain constructs needed to be subcloned into mammalian expression vectors. The vectors (pEE6.1, pEE12.1, pEE14.1) contain the promoter, poly-A signal and other sequences necessary for expression in mammalian cell lines. The chimerised constructs were digested with HINDIII/EcoRI and subcloned into pEE6.1 (heavy) and pEE12.1/pEE14.1 (light) using the same restriction enzyme sites.

FIG. 4 shows chimeric LOB 7/4H in pEE6.1, chimeric LOB 7/4 K in pEE12.1 or pEE14.1 all digested with HINDIII and EcoR1 restriction enzymes.

Stable expression transfectants can be obtained by co-transfecting CHO-K1 cells with the chimeric heavy and light chains into separate vectors. However, it is more convenient to have the 2 chimerised chain within 1 plasmid vector. This was achieved by digesting the chimerised heavy chain expression cassette in pEE6.1 with Notl/BamHI enzymes and ligating into PEE12.1/pEE14.1 plasmids containing the chimerised light chain via the same restriction sites. The resulting plasmid would contain both chimerised heavy and light chains in one expression cassette.

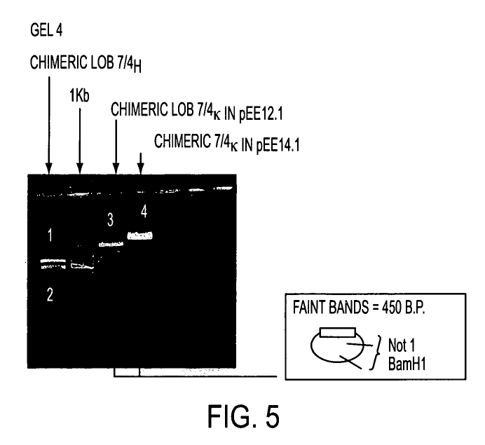

FIG. 5 shows chimeric LOB 7/4H in pEE6.1 and chimeric LOB 7/4K in pEE12.1 or 14.1 all digested with NOT1 and BamH1

The DNA was extracted, ligated and transformed as above to produce the chimeric heavy and light chains within one plasmid. The plasmid was then transfected into CHO-K1 cells using the Gene-Porter technique.

Transient expression and subsequently stable expression of the human chimeric anti-CD40 antibody (chLOB 7/4) was identified using ELISA and indirect FACS analysis.

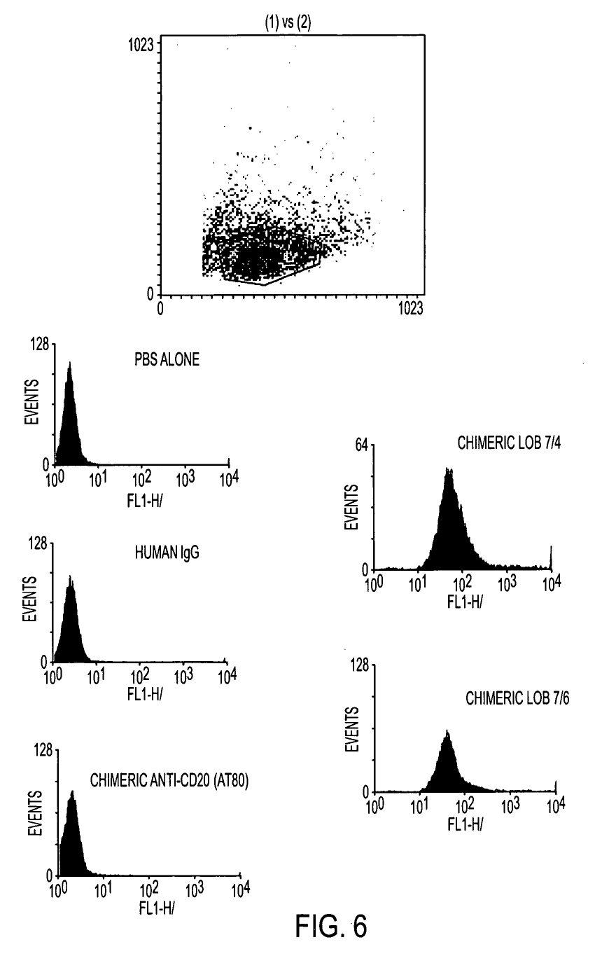

FIG. 6 contains an indirect flow cytometric analysis of murine B cells transfected to express human CD40 and used to confirm the presence of the chimeric human anti-CD40 monoclonal antibody in the supernatant obtained from the transfected CHOK1 cells.

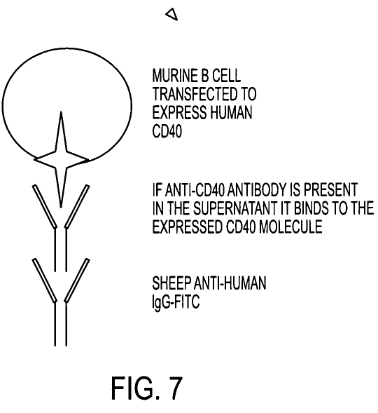

FIG. 7 contains a schematic to represent the method of indirect flow cytometric analysis used to confirm the presence of chimeric human anti-CD40 antibody (ch LOB 7/4)

The chimeric LOB 7/4 was purified on a 1.5 ml protein-A sepharose column equilibrated with 40 mM tris/HCL, 2 mM EDTA and 200 mM sodium chloride buffer at a pH of 9.0. Initially 1.6 liters of supernatant was passed through the column with 50 ml of the tris buffer. The peak was eluted with glycine 200 mM/EDTA buffer 2 mM at a pH of 3.0. The antibody was dialyzed against PBS. The optical density was recorded as 0.9, equivalent to a concentration of 0.67 mg/ml. The presence of the antibody was confirmed by gel electrophoresis. The supernatant was then reloaded onto the column and re-eluted with the glycine buffer. The eluted solution was dialyzed against PBS and pooled with the previous eluent. The optical density was recorded as 0.662, equivalent to 0.49 mg/ml of antibody. The total quantity of antibody obtained from 1.6 L of supernatant was 9.8 mg contained in 20 mls of PBS.

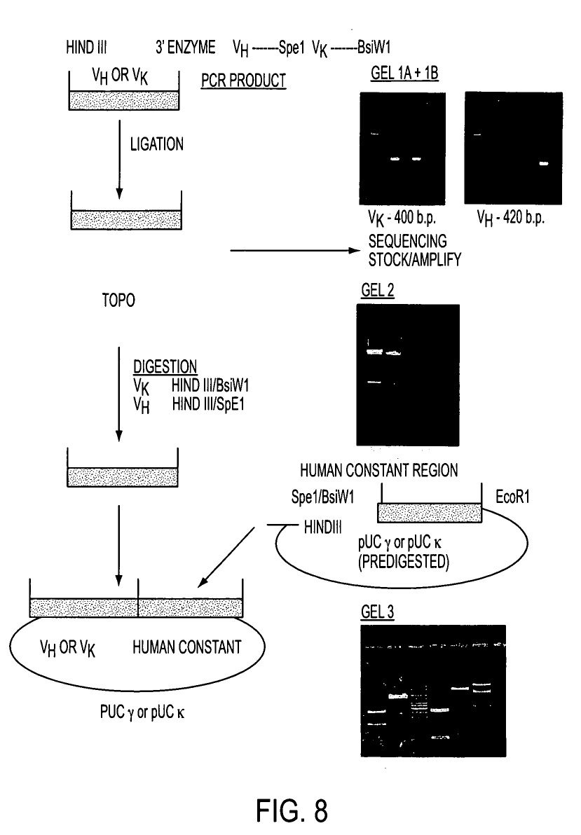

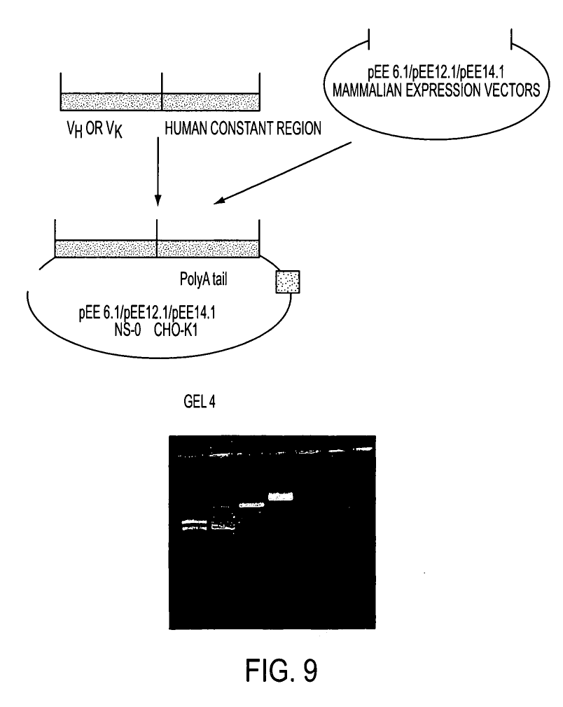

FIG. 8 shows initial stages in the production of human chimeric anti-CD40 (ch LOB 7/4) FIG. 9 depicts schematically the insertion of LOB 7/4 heavy and light chains in an mammalian expression vector system.

### Example 2

### Costimulatory Assays

LOB 7/4 the parent antibody of chimeric LOB 7/4 was assessed against a known agonistic anti-CD40 antibody mAb s2c6 for its ability to upregulate the key costimulatory molecules B7.1 and B7.2 in a dendritic cell culture system developed by Jan Fisher and Chris Treter of Dartmouth Medical Center. LoB 7/4 was found to upregulate B7.1 and B7.2 in greater than 90% of cultured dendritic cells, similar to the s2c6 antibody. [Harvey et al., “CD40 Antibodies for the treatment of human malignancy” 2002]

### Example 3

### Growth Inhibitory Assays

Light microscopy of beads linked to LOB 7/4 or chimeric LOB 7/4 and incubated with CD40 expressing cells (Daufdi) showed obvious antibody mediated bead-cell binding. Beads linked to negative control mAbs (DB7-18 or Irr Hu IgG did not bind to CD40 expressing cells.

Growth inhibition of human non-Hodgkin's lymphoma cell lines was assessed using [3H methyl} thymidine incorporation assays. Incubation of RL and Daudi cell lines for 5 days with LOB 7/4 and chimeric LOB 7/4 led to significant inhibition of cellular proliferation when compared to incubation with irrelevant, isotype matched murine (DB7-18) or human (Irr Hu IgG) mAb to cells incubated without antibody. All antibodies were presented linked to microbeads; (100 micrograms linked to 2×10 8 beads). Maximal growth inhibition occurred at a bead concentration of 50,00 beads per well.

Growth inhibition of human epithelial cancer cell lines was assessed using the tetrazolium bromide conversion assay. Incubation of EJ138 and Caski cell lines for 5 days with LOB 7/4 and chimeric 7/4 led to a significant inhibition of cellular proliferation when compared to incubation with irrelevant, isotype matched murine (DB7-18) or human (Irr Hu IgG)mAb or to cells without antibody. All antibodies were presented linked to microbeads; (100 micrograms linked to 2×10 8 beads). Maximal growth inhibition occurred at a bead concentration of 500,000 beads per well.

### Example 4

### Complement Mediated Cytotoxity

a. Human Non-Hodgkin's Lymphoma Cell Lines

The ability of LOB 7/4 and chimeric LOB 7/4 to mediate complement mediated cytotoxity (CDC) was assessed using the CDC chromium 51 release assay. Chimeric LOB 7/4 was able to induce significant CDC (as measured using the specific chromium 51 release) in both RL and Daudi cells. In RL cells, chimeric LOB 7/4 specific chromium 51 release was maximal (22%) at a final antibody concentration of 0.4 micrograms per ml. In daudi cells, chimeric LOB 7/4 specific chromium 51 release was maximal (65%) at a final antibody concentration of 2 micrograms per ml. LOB 7/4 did not mediate effective CDC.

b. Human Epithelial Cancer Cell Lines

Effective chimeric LOB 7/4 mediated CDC could not be demonstrated in EJ138 or MG79 cell lines.

### Example 5

### Antibody Directed Cellular Cytotoxicity

a. Human non-Hodgkin's Lymphoma Cell Lines

The ability of LOB 7/4 and chimeric 7/4 yto mediate antibody mediated cellular cytotoxicity (ADCC) was assessed using an ADCC chromium 51 release assay. Chimeric LOB 7/4 was able to induce significant ADDCC (As measured by specific chromium 51 release) in both RL and Daudi cells. In RL cells, maximal chimeric LOB 7/4 mediated specific chromium release (65%) was seen at a final antibody concentration of 10 micrograms/ml and an effector:target ratio of 50:1. Chimeric LOB 7/4 mediated specific chromium 51 release (71%) was seen at an antibody concentration of 10 micrograms/ml and at an effectors target ratio of 50:1. LOB 7/4 did not mediate effective ADCC in either cell line.

b. Human Epithelial Cell Lines

Effective chimeric LOB 7/4 mediated ADCC. could not be demonstrated in EK138 or MG79 cell lines

Flow cytometric confirms the expression of CD40 on the surface of human non-Hodgkin's cell lines RL and Daudi and the epithelial cancer cell lines EJ138 and Caski. Ligation of surface CD40 by LOB 7/4 or chimeric LOB 7/4 presented on M-450 Dynabeads caused significant growth inhibition in the human NHL cell lines RL and Daudi and in the malignant epithelial cell lines EJ138 and Caski. Binding of Chimeric LOB 7/4 to surface CD40 on RL and Daudi cells was able to effectively activate complement and mediate CDC. This effect could not be reproduced in human epithelial cell lines. Due to its murine Fc domain, LOB 7/4 was unable to mediate CDC with human complement. Binding of LOB 7/4 to surface CD40 of RL and Daudi cell lines was able to effectively mediate ADCC. Due to its murine Fc domain, LOB 7/4 did not mediate significant ADCC with human effector cells. These results suggest that chimeric LOB 7/4 is able to bind in a similar manner to the parent LOB 7/4 antibody and is functional. The human constant regions may allow the chimeric antibody to effectively interact with human effector mechanisms.

### Example 6

### Dosing Studies

a. Multiple Dosing Studies

Animals received a total of four weekly treatments of up to 100 micrograms of chimeric LOB 7/4 by IV or IP injection. These animals remained well throughout the course of their treatment regardless of dose and there were no animal deaths in any treatment group.

b. High Does Single Dose Studies

Six pathogen free C57BLK/6 mice and six Syrian hamsters were administered a single 10 mg IP dose of endotoxin free chimeric LOB 7/4. Animals did not appear unwell following antibody injection and continued to feed normally and gain weight. Terminal bleeds were performed for biochemical and hematological analysis and results compared to untreated control animals. No significant biochemical or hematological abnormalities were seen. No macroscopic abnormalities were seen at post-mortem. Given the toxicities observed in 3/23 treated animals, specimens of liver, kidney and spleen were taken from each animal, preserved in formalin and processed into paraffin blocks. Slides cut from each of these blocks were stained with hematoxylin and eosin; no significant histopathological abnormalities were observed in any tissue examined. Additionally, six C7BLK/6 mice and six Syrian hamsters received 10 mg IP of chimeric LOB 7/4 without hematological, biochemical or histopathological evidence of toxicity.

### Example 7

### Expression of CD40 on Normal Human Cells

a. Normal Human Tissue Samples

A wide range of human tissues were selected for evaluation of normal CD40 expression. LOB 7/6 yielded consistently good results at a working dilution of between 1:600 and 1:1000. These results showed that cells derived from tonsil (B cells, macrophages), lymph node (B cells, macrophages), spleen (B cells, macrophage), liver (inflammatory cell infiltrate), uroepithelial tract (transitional cell urothelium) skin (inflammatory cells), colon (B cells), stomach (B cells), lung (alveolar macrophages), small intestine (macrophages), and parotid (inflammatory cells) stained positively. By contrast, kidney, uterus, muscle, ovary, thyroid, pancreas, salivary gland and brain cell did not.

b. Malignant Human Tissue Samples

A selection of malignant human tissues were selected for evaluation of tumor CD40 expression. LOB 7/6 yielded consistently good results at a working dilution of between 1:600 and 1:1000. CD40 positivity was demonstrated in a range of paraffin embedded B cell non-Hodgkin's lymphoma and solid tumor specimens. Particularly tissue sections from 3 diffuse large B cell lymphoma showed positive CD40 expression in all tested sections. Tissue sections obtained from 3 patients with melanoma revealed positive CD40 expression in 2 out of 3 tested sections. Tissue sections from 11 patients with no-small lung carcinoma showed CD40 expression in 5/11 patient sections. Tissue sections from a single patient with invasive ductal breast carcinoma showed positive CD40 expression. Tissue sections from 4 patients with renal cell carcinoma did not show CD40 expression in all 4 samples. Tissue sections from 3 patients with colorectal adenocarcinoma did not reveal CD40 expression in any of the 3 samples. Tissue sections from 3 patients with transitional cell carcinoma bladder did not reveal any CD40 positive expression.

### Example 8

### Detection of LOB 7/4 in Human Serum

To ensure chimeric LOB 7/4 could be detected reliably in human serum, three human serum samples were spiked with chimeric LOB 7/4 20 micrograms/ml, diluted 1:10 to 1:16,000 and evaluated by ELISA. Spiked serum was compared to unspiked serum and a chimeric LOB 7/4 standard curve (in PBS/BSA alone) at a identical dilution. The addition of serum did not influence the detection of LOB 7/4. Chimeric LOB 7/4 was reliably detected at the lowest concentration evaluated (1.25 micrograms/ml).

### Example 9

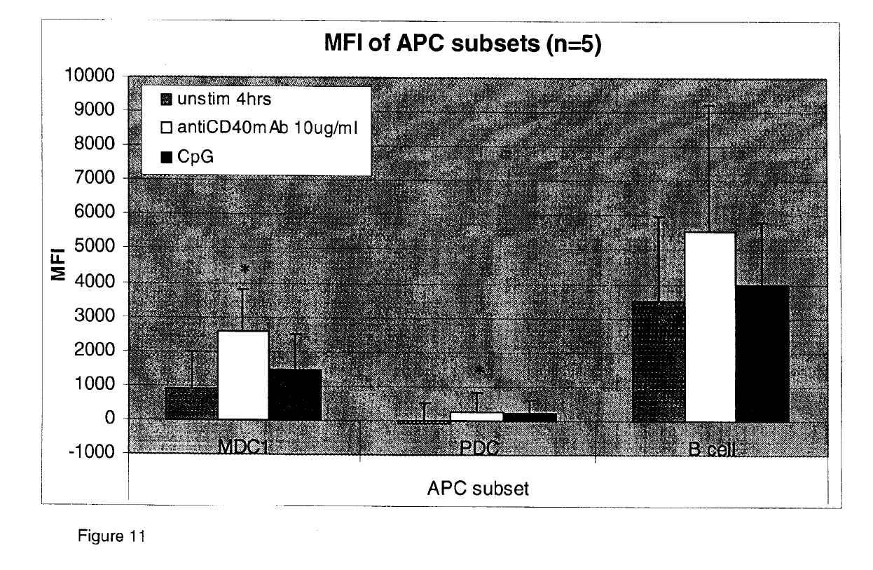

This example relates to the experiment contained in FIG. 11. In this experiment FACS analysis of the early activation marker CD83 was effected on APC subsets (MDC1 myeloid dendritic cells, PDC plasmacytoid dendritic cells and B cells) after whole blood incubation for 4 hours at 37° C./5% CO2, either unstimulated or stimulated with the subject chimeric anti-CD40 mAb or CpG oligonucleotide.

As can be seen from the results in FIG. 11, this FACS analysis revealed a significant increase in CD83 expression on MDC1 and PDC subsets after anti-CD40 mAb stimulation in comparison to unstimulated (p<0.05 determined using paired ttest)

### Example 10

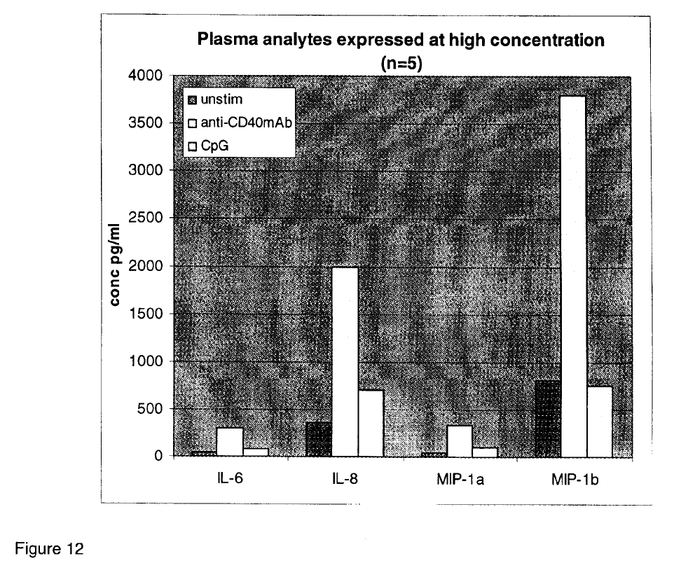

This example relates to the experiment contained in FIG. 12. In this experiment plasma removed from whole blood samples following stimulation with/without the subject chimeric anti-CD40 mAb and CpG oligonucleotides was analysed using a multiplex cytokine array panel (Luminex) to determine levels of 10 cytokine/chemokines to assess changes in the plasma cytokine profile.

As shown in FIG. 12, this Luminex analysis revealed high concentrations of IL-6, IL-8 MIP-1α and MIP-1b being expressed after anti-CD40 mAb stimulation.

### Example 11

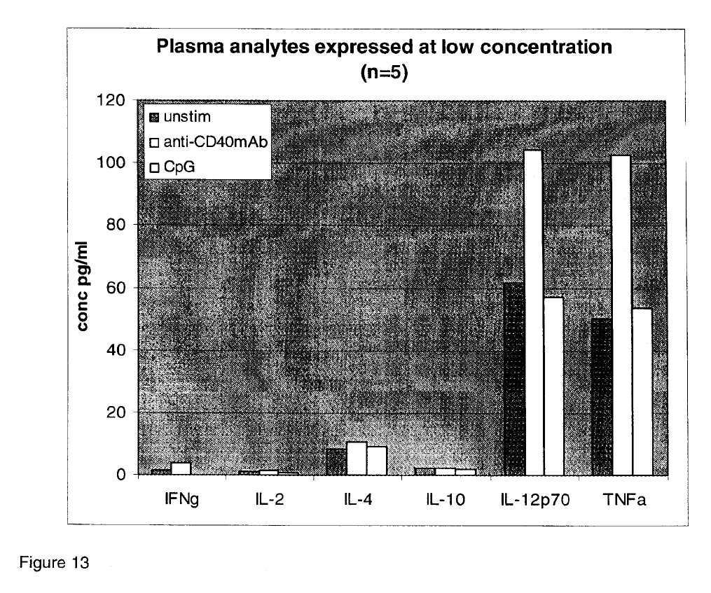

This example relates to the experiment contained in FIG. 13. In this experiment plasma was removed from whole blood samples following stimulation with/without the subject chimeric anti-CD40 mAb and CpG oligonucleotides was analysed using a multiplex cytokine array panel (Luminex) to determine levels of 10 cytokine/chemokines to assess changes in the plasma cytokine profile.

As can be seen from FIG. 13, this Luminex analysis revealed there to be low concentrations of IL-12p70 and TNFa being expressed after stimulation with the inventive chimeric anti-CD40 mAb.

### CONCLUSIONS

Anti-CD40 mAb therapy in a murine using an antimurine CD40 antibody (3/23) has shown that 3/23 treatment results in a dose dependent acute hepatitis, peaking in severity at 1-3 weeks following a single intraperitoneal injection but recovering fully around week 5. Detailed histochemical analysis revealed a widespread acute lymphogranulomatous hepatitis progressing yto piecemeal necrosis in the most affected livers. The underlying mechanism of this antibody (3/23) mediated hepatatic damage is unclear. Interestingly, the dose limiting toxicity of s published phase I clinical study in humans using an anti-CD40L antibody was hepatic transaminitis akin to the results seen in the comparative mouse model using 3/23. A possible mechanism is the interaction of Fas and Fas ligand. This death receptor-ligand pair are members of the TNF family. FasL is expressed on activated cytotoxic T cells and is important in mediating cellular cytotoxicity through cross-linking of Fas receptor on liver cells. The liver has been shown to be a site for clearance of lymphocytes and as such is an organ at potential risk of damage by infiltrating “inappropriately activated” cytotoxic T lymphocytes. Large numbers of CD8 positive lymphocytes were apparent in livers of the 3/23 treated mice.

Also, splenomegaly was noted in all animals receiving 1 mg or more of 3/23. Spleen size increased significantly (up to six fold increase in weight) within 1-2 weeks of 3/23 treatment but returned to normal by week 8. Histopathological examinations of enlarged spleens revealed hypertrophy of the per arteriolar lymphoid sheaths and marginal zones. These results are consistent with B and T cell activation and proliferation secondary to CD40 crosslinking.

A noted above, these results wee not seen in the dose studies in CB7BLK/6 mice and Syrian hamsters administered the chimeric LOB 7/4. This is not unexpected because this antibody contains a human constant region and a variable region that does not target a murine antigen.

Similar responses to multiple dosing studies treated with monoclonal anti-CD40 antibodies have been observed by other groups and attributed to cytokine release from stimulated CD40 positive cells. (Melief et al., Immunol. Rev. 188:177-182 (2002)) Alternatively, this may be attributable to an acute anaphylactic response to the antibody. (augmented by the immunostimulatory properties of anti-CD40 therapy), rather than an acute response to the CD40 crosslinking.

When LOB 7/4 was presented by the low affinity human Fc receptor, expressed by a feeder layer of transfected mouse fibroblasts (Fcγ RII/CDw32), significant growth inhibition was just achieved in the RL system. This was not identified when LOB 7/4 was substituted with some of our other mouse anti-human CD40 antibodies (LOB 7/2, 7/6, 7/8). There are several reasons why LOB 7/4 might be a better inducer of growth inhibition. Possible explanations include steric orientation, agonist activity, affinity, avidity, and the different epitopes of the antibodies.

CD40 ligation with human SCD40L, in vitro, caused significant growth inhibition with both ovarian and cervical cell lines (MG79 and Caski respectively).

It is known that cross-linking, in vitro, markedly enhances the inhibitory signals of anti-CD40 antibodies [Atkins yet. al, Canc J. Sci. Amer. 3 Suppl. 1:p S7-8 (1997)]. Experiments have been performed with Burkitt's lymphoma cell lines showing that soluble anti-CD40 does not significantly inhibit cell growth but immobilized anti-CD40 does. In addition, anti-CD40 significantly prolongs the life of mice bearing this tumor. This explains the different growth inhibitory effects seen when RL and daudi cells were cultured with TCHO, SCD40L and anti-CD40 antibody alone. However, it is more difficult to explain the different observations seen when these same cells were cultured with Fcγ RII/CDw32 cells and anti-CD40 monoclonal antibody. This system should provide appropriate cross-linking of the antibody, yet in my experiments growth inhibition was not clearly apparent and only just achieved significance with LOB 7/4. The relative insensitivity of these particular Fcγ RII/CDw32 cells to the effects of radiation, delivered to prevent cellular proliferation, was problematic but even with successful irradiation the proliferation of the B cells was not influenced with the addition of anti-CD40 antibody. The results were variable and difficult to assess in this more complicated system. Further work should include pre-incubation of the B cells with polyclonal human IgG to block non-specific antibody binding sites. A new line of FCγ RII/CDw32 cells could be produced and compared with the original ‘boosting’ the immune response to tumor. Activated tumor cells could also be used to stimulate the ex-vivo production of autologous human T-cells, these could be adoptively transferred. The potential effects of widespread CD40 ligation would then be avoided.

It is to be understood that the invention is not limited to the embodiments listed hereinabove and the right is reserved to the illustrated embodiments and all modifications coming within the scope of the following claims.

The various references to journals, patents, and other publications which are cited herein comprise the state of the art and are incorporated by reference as though fully set forth.

1. A method of treating human cancer which comprises administering a therapeutically effective amount of a anti-human CD40 antibody selected from:
(i) an anti human CD40 antibody containing the consensus sequences of LOB 7/4 encoded by the nucleic acid sequences contained in FIG. 3 or a variant thereof which has been humanized to avoid immunogenicity in humans;
(ii) an anti human CD40 antibody containing the complementarity determining regions of LOB 7/4 encoded by the nucleic acid sequences contained in FIG. 3; and
(iii) an anti-human CD40 antibody which competes with and/or binds the same human CD40 epitope as LOB 7/4.

2. The method of claim 1 which is used to treat a CD40 expressing cancer.

3. The method of claim 2 wherein said cancer comprises a solid tumor.

4. The method of claim 1 wherein said cancer is selected from the group consisting of: breast, liver, ovarian, colorectal, lung, stomach, kidney, melanoma, ovarian and non-Hodgkin's lymphoma.

5. The method of claim 1 wherein the administered antibody comprises LOB 7/4 or a humanized variant thereof.

6. The method of claim 1 wherein the treated cancer is a lymphoma.

7. The method of claim 6 wherein said lymphoma is a non-Hodgkin's lymphoma.

8. The method of claim 1 wherein the treated cancer is renal cancer.

9. The method of claim 1 wherein said anti-human CD40 antibody is administered in combination with at least one chemotherapeutic.

10. The method of claim 1 wherein said antibody is administered in combination with at least one cytokine or other immune agonist molecule.

11. The method of claim 10 wherein said cytokine is an interferon, interleukin, tumor necrosis factor or colony stimulating factor.

12. The method of claim 10 wherein the cytokine is alpha or beta interferon.

13. The method of claim 10 wherein the immune agonist is a TLR agonist.

14. The method of claim 13 wherein the TLR agonist is a TLR1, TLR2, TLR3, TLR4, TLR5, TLR6, TLR7, TLR8, TLR9, TLR10 or TLR11 agonist.

15. The method of claim 13 wherein the TLR agonist is a flagellin, CPG oliogo, or polyIC.

16. The method of claim 12 wherein said administration of the antibody and interferon is effected separately, in either order, or in combination and elicits a synergistic effect on immunity.

17. The method of claim 13 wherein the administration of the antibody and the TLR agonist is effected separately, in either order, or in combination, and elicits a synergistic effect on immunity.

18. The method of claim 1 wherein said antibody is conjugated to a chemotherapeutic agent or cytokine or TLR agonist.

19. The method of claim 18 wherein the cytokine is an alpha interferon or beta interferon.

20. The method of claim 18 wherein the TLR agonist is a flagellin.

21. The method of claim 1 wherein said immunoglobulin is a chimeric or humanized immunoglobulin.

22. The method of claim 1 wherein said immunoglobulin comprises human heavy and light chain constant regions.

23. The method of claim 22 wherein said immunoglobulin is selected from the group consisting of an IgG1, IgG2, IgG3 and an IgG4.

24. The method of claim 1 wherein said antibody is administered in conjunction with another therapeutic antibody that binds to a different antigen.
